(19) United States
(12) Patent Application Publication
(10) Pub. No.: US 2026/0192060 A1
(43) Pub. Date: Jul. 9, 2026

(54) MEMBRANE FOR A VIBRATING MESH MODULE

(71) Applicant: Philip Morris Products S.A., Neuchatel (CH)

(72) Inventors: Dara Zaman BAYAT, Neuchâtel (CH); Leander DITTMANN, Crissier (CH); Dino FARALLI, Grandson (CH); Laura GALLIMBERTI, Morges (CH)

(73) Assignee: Philip Morris Products S.A., Neuchatel (CH)

(21) Appl. No.: 19/131,983

(22) PCT Filed: Dec. 14, 2023
(86) PCT No.: PCT/EP2023/085781

§ 371 Date: May 22, 2025

(30) Foreign Application Priority Data
Dec. 14, 2022 (EP) 22213495

(51) Int. Cl. A61M 11/00; A61M 15/00; B05B 17/06; B06B 1/06

(52) U.S. Cl. CPC A61M 11/005; A61M 15/0005; B05B 17/0607; B06B 1/0651; B06B 1/0666; A61M 2205/0294; A61M 2207/00; B06B 2201/77

(57) ABSTRACT

A membrane for a vibrating mesh module for an aerosol-generating device is provided, the membrane including: a plurality of recesses defining portions of the membrane having reduced thickness, the portions of the membrane having reduced thickness being provided with one or more through-holes defining perforations of the membrane; and one or more piezo elements integrally formed therewith, the through-holes having an aspect ratio of below 1, and the through-holes having a diameter of between 0.2 micrometer and 3 micrometers.

The present invention relates to a membrane for a vibrating mesh module for use in an aerosol-generating device. The present invention also relates to an aerosol-generating device comprising such vibrating mesh module. The present invention also relates to a method of manufacturing a membrane for a vibrating mesh module for use in an aerosol-generating device.

Aerosol-generating devices using vibrating mesh modules are often also referred to as vibrating mesh (VM) nebulizers. Such vibrating mesh nebulizers are used for the generation of a respirable aerosol that can be used for instance for treating respiratory diseases.

Vibrating mesh nebulizers use mesh deformation or vibration to push a liquid through a mesh. In a typical vibrating mesh nebulizer, a piezo element, which is in contact with a mesh, is used to produce vibrations of the mesh. The mesh is adjacent and in direct contact with a liquid drug-containing substrate. Holes in the mesh may have a conical structure, with the largest cross-section of the cone in contact with the liquid substrate. The mesh deformation generates a pressure field in the liquid, thus pumping and loading the holes with liquid. The liquid volume displaced through the holes breaks up into droplets, and are ejected into a mouthpiece chamber. The droplets mix with the air and directly form an aerosol in the mouthpiece chamber. During inhalation, ambient air crosses the mouthpiece chamber to transport the aerosol to the user.

Several off-the-shelves vibrating mesh nebulizer devices are available today. These devices comprise vibrating mesh modules that may mainly consist of two parts: An annular piezo-element and a circular, perforated membrane. In case the membrane is formed from metal or stainless steel, the perforations are typically created by laser drilling. Laser drilling leads to conical holes having a small cone angle and a comparably high aspect ratio. Membranes being made from nickel or a nickel alloy, are typically perforated by a combined lithography and electroplating process. In a first step, such membranes are perforated using a lithographic process to obtain a desired pattern of holes with a comparatively large diameter. Then the entire membrane undergoes electroplating to deposit material on the surface thereby shrinking the size of holes. The shape of the resulting holes is funnel-like, which is generally more preferred with respect to their microfluidic properties. In addition, this process may result in a lower aspect ratio and is therefore also for this reason preferred over conically shaped laser drilled holes.

The size of the holes of the membranes used in either of these commercially available devices is limited to about 3 to 4 micrometers. Therefore, the MMAD (Median Mass Aerodynamic Diameter) of the aerosol size distribution obtainable by such devices is also in the range of 3 to 4 micrometers, which is too big for deeper lung inhalation. Accordingly, most of the aerosol will be deposited in the upper region of the respiratory tract, which may be disadvantageous for efficient drug uptake and may cause throat irritation.

Reduction of the dimension of the holes in conventional membranes may not be possible, since a decreasing hole diameter requires an increased pressure to push the liquid through the hole. This increase could be compensated by reducing the aspect ratio (length of the hole divided by diameter of the hole) of the holes. However, due to mechanical stability reasons the thickness of the membranes cannot be arbitrarily reduced in conventional manufacturing methods. Further, the liquid throughput rate per hole decreases quadratically with decreasing hole diameter. Thus, in order to maintain a desired aerosol throughput such membranes would have to be provided with a significantly higher number of holes.

Finally, standard manufacturing techniques for VM modules may require a couple of cumbersome assembly steps, including gluing the piezo actuators to the perforated membrane. If the gluing is not properly done the VM module will have low energy conversion efficiency and may even lead to damage during operation. The glue may also not be compatible with some liquids used with the VM module, bearing the risk of liquid contamination and would require additional protection means.

It would be desirable to provide a perforated membrane for a vibrating mesh module overcoming at least one of the abovementioned drawbacks.

It would be desirable to provide a perforated membrane allowing to generate aerosol particles having a reduced diameter while at the same time maintaining the volume of the generated aerosol.

It would further be desirable to provide a manufacturing method that allows to reliably and reproducibly manufacture perforated membranes with specific aerosol-generating properties. In particular it would be desirable to provide a method to manufacture vibrating mesh modules without the need of glue or a gluing step.

In summary, there is a need in the art to manufacture membranes for VM modules with reduced exit hole diameter, decreased aspect ratio of the through holes, increased number of holes, and avoiding glue to assemble the module.

According to an embodiment of the invention there is provided a membrane for a vibrating mesh module for use in an aerosol-generating device. The membrane comprises a plurality of recesses, which recesses define portions of the membrane having reduced thickness h. The portions of the membrane having reduced thickness h are provided with one or more through-holes to define perforations of the membrane.

By providing a membrane comprising recesses, portions of the membrane are defined, which have a reduced thickness. The overall structural stability of such membrane is maintained by the non-recessed portions of the membrane. At the same time, the recessed portions having a reduced thickness allow to define therein through-holes with diameters that are sufficiently small to generate aerosol droplets for deep lung inhalation.

The through-holes formed in the recessed portions may have a diameter of between 0.1 and 4 micrometers. The through-holes formed in the recessed portions may have a diameter of between 0.2 and 3 micrometers.

The diameter of the through-holes on the membrane is a key parameter to define the resulting aerosol droplet size. The smaller this diameter, the smaller the droplets formed upon aerosolization. The through-hole diameter of conventional membranes manufactured with currently available techniques is limited to about 3 to 4 micrometers. Correspondingly, the median mass aerodynamic diameter (MMAD) of the aerosol particle size distribution is in the range of 3 to 4 micrometers. In contrast thereto, the membranes described herein have through-holes with smaller diameters and therefore allow for smaller MMAD of the aerosol particle size distribution.

The through-holes in the membrane may have an aspect ratio of below 3. The through-holes may have an aspect ratio of below 1. The aspect ratio of a through-hole may be defined as the length of the through-hole divided by its diameter. Accordingly, for the through-holes provided in the recesses of the membrane, the aspect ratio is given by the reduced thickness of the membrane at the recesses divided by the diameter of the through-holes. Small aspect ratios may be beneficial. The smaller the aspect ratio, the smaller the pressure that is required to push the liquid through the through-holes.

The through-holes may have any hole geometry. The through-holes may have a circular cross-section. A circular cross-section provides the highest ratio of open cross-sectional area to boundary surface area, which is beneficial to reduce microfluidic flow resistance through the holes. However, the through-holes may be formed to have any other desired cross-sectional shape. For the sake of simplicity, with regard to the lateral dimension of the through hole it is herein often referred to the diameter of the through-holes. In case of through holes with non-circular cross section, the term diameter shall be interpreted as to refer to the largest lateral dimension of the through-holes.

The thickness of the non-recessed portions of the membrane may be significantly larger than the residual thickness of the membrane at the recesses. The membrane may have a thickness of 5 micrometers to 500 micrometers. The membrane may have a thickness of 10 micrometers to 400 micrometers. The membrane may have a thickness of 30 micrometers to 200 micrometers. The thickness of the membrane significantly determines mechanical behaviour of the membrane. Accordingly, the thickness of the membrane may be chosen depending on the targeted operation frequency and properties of the membrane material.

As explained above, the membrane is provided with recesses defining portions of the membrane having reduced thickness. The recesses themselves may have any desired cross-sectional shape. The recesses may have a circular cross-sectional shape. The diameter of the recesses may range from 5 micrometers to 300 micrometers. The diameter of the recesses may range from 10 micrometers to 200 micrometers. The diameter of the recesses may range from 15 micrometers to 100 micrometers.

With the above-described geometrical dimensions, recesses may be formed covering a broad range of aspect ratios. It is preferred, however, to form the recesses such that they have an aspect ratio of below 3. For the aspect ratio of the recesses, the same is true as described above in relation to the aspect ratio of the through-holes. The smaller the aspect ratio of a recess, the less pressure is required to push liquid through the recess.

Each recess is provided with one or more through-holes. The number of through-holes may depend on the size of the recesses and the size of the through-holes. A recess may comprise from 1 to 1500 through-holes. A recess may comprise from 10 to 1000 through-holes.

The overall membrane may comprise a density of through-holes of between 10 and 10000 through-holes per squaremillimeter. The overall membrane may comprise a density of through-holes of between 50 and 5000 through-holes per squaremillimeter.

Details of the final configuration of the membrane may be chosen depending on the liquid substrate that is to be aerosolized. Details of the final configuration of the membrane may be chosen depending on the desired liquid throughput.

The liquid throughput per through-hole largely depends on the diameter and on the open surface area of each through-hole. Accordingly, the liquid throughput of a through-hole decreases quadratically with decreasing hole diameter. Thus, in order to maintain a desired throughput of a membrane with smaller through-holes, the number of through-holes in a membrane has to be increased correspondingly. In addition, the aspect ratio of the through-holes needs to be reduced in order to maintain the pressure to push the liquid through the through-holes at a sufficiently low level. Thus, for the final configuration of the membrane these factors may have to be taken into account. The final configuration may also need to be adapted to the properties of the liquid to be aerosolized. The configuration may particularly depend on the viscosity of the liquid to be aerosolized.

The membrane may be manufactured by making use of micro-electro-mechanical systems (MEMS) manufacturing techniques. Such manufacturing techniques include process technologies used in semiconductor device fabrication. These techniques include deposition of material layers, patterning by photolithography and etching of material in order to produce the required shapes.

Accordingly, the membrane may be made of material suitable to be processed by MEMS manufacturing techniques. The membrane may comprise a bulk layer formed from a material provided in the form of a wafer. The wafer material may be made of silicon, silicon oxide, silicon nitride, aluminium nitride, or combinations thereof.

The membrane structure may comprise a plurality of additional layers of material. These layers may be applied to the bulk wafer by thin film deposition techniques.

MEMS technology allows to tailor the hole geometry and shape to meet the needs required by the application. As discussed above, this includes in particular the diameter of the through-holes, which is a crucial parameter to adjust the resulting aerosol droplet size. MEMS technology also allows to define the aspect ratio of the through-holes, which is an important parameter to enable and control the microfluidic flow through the through-holes. As will be discussed in more detail below, the MEMS manufacturing process may include a plurality of photolithographic masking and etching steps. The lateral size of the mask defines the lateral placement and sizes of recesses and through-holes in the membrane structure and can easily be adapted to specific needs. The combination of etchant, wafer material, and wafer lattice orientation allows to define the cross-sectional shape of the etched recess and the depth of the resulting recesses. Subsequent etching steps thus allow the manufacturing of complex shapes of the recesses and through-holes of the membrane structure.

Etching might be accomplished by wet etching or dry etching or a combination thereof. Dry etching, for example reactive ion etching or plasma etching, is preferred as it results in vertical edges independent of the lattice orientation. While in contrast most wet etching techniques are anisotropic and the resulting patterns are not only defined by the mask but also by the lattice orientation, for example silicon in <100> orientation with a KOH etching results in pyramidal shapes through the thickness of the membrane with an angle defined by the lattice plane orientation

The invention further relates to an aerosol-generating device with a vibrating mesh module comprising a membrane as described herein. As used herein, the term “aerosol-generating device” refers to a device that interacts with an aerosol-forming substrate to generate an aerosol. An aerosol-generating device may interact with one or both of an aerosol-generating article comprising an aerosol-forming substrate, and a cartridge comprising an aerosol-forming substrate. An aerosol-generating device may comprise a housing, electric circuitry including a controller, a power supply and a plurality of sensors.

As used herein, the term “aerosol-generating system” refers to the combination of an aerosol-generating device with an aerosol-forming substrate. When the aerosol-forming substrate is provided in a cartridge, the aerosol-generating system refers to the combination of the aerosol-generating device with the cartridge. In the aerosol-generating system, the aerosol-forming substrate and the aerosol-generating device cooperate to generate an aerosol.

The invention further relates to a method of manufacturing a membrane for a vibrating mesh module for use in an aerosol-generating device. The method comprises:
providing a bulk wafer of a first material, the bulk wafer having opposing first and second surfaces,
depositing a cover layer of a second material onto the first surface of the bulk wafer,
providing through-holes to the cover layer using MEMS manufacturing techniques,
etching the second surface of the bulk wafer to define recesses therein,
etching the second surface of the bulk wafer until the bulk waver material is removed from the recesses,
cutting out the membrane.

The method of manufacturing the membrane may comprise a plurality of manufacturing steps, which are also used in MEMS manufacturing and which may include processes such as deposition of material layers, patterning by photolithography and etching of material in order to produce the required shapes.

Thin film deposition may be achieved by physical vapour deposition (PVD) including techniques such as evaporation, magnetron sputtering or pulsed laser deposition (PLD). These techniques can be used to deposit one or more layers of material in a desired sequence onto the bulk wafer.

Photolithography is a well-known technique, in which light is used to produce minutely patterned thin films of suitable materials over a bulk substrate, to protect selected areas of the substrate during subsequent etching, deposition, or implantation operations. Typically, ultraviolet light is used to transfer a geometric design from an optical mask to a light-sensitive chemical (photoresist) coated on the substrate. The photoresist either breaks down or hardens where it is exposed to light. The patterned film is then created by removing the softer parts of the coating with appropriate solvents.

The etching steps might be accomplished by wet etching or dry etching or a combination thereof. Dry etching, for example reactive ion etching or plasma etching, may be preferred, since it may be used to create vertical edges in a substrate, independent of the crystallographic orientation of the substrate. Wet etching techniques may also be used. However, these techniques are usually anisotropic and the resulting patterns are not only defined by the previously applied mask, but may also depend on the lattice orientation. For example, silicon in <100> orientation with a KOH etching results in pyramidal shapes through the thickness of the membrane with an angle defined by the lattice plane orientation.

The bulk wafer may be made of any first material that is suitable to be processed by MEMS manufacturing techniques. The bulk wafer material may be made of silicon, silicon oxide, silicon nitride, aluminium nitride, or combinations thereof. The materials of the one or more additional layers applied to the bulk wafer may also be selected from any material that is suitable to be processed by MEMS manufacturing techniques. The materials of the one or more additional layers applied to the bulk wafer may be selected from silicon, silicon oxide, silicon nitride, aluminium nitride, or combinations thereof. In addition, the additional layers may be formed from materials having specific functional properties, such as selective etching stoppers or materials enhancing flow properties of the liquid substrate that is to be aerosolised.

In the method of manufacturing a membrane for a vibrating mesh module for use in an aerosol-generating device a bulk wafer of a first material such as silicon is provided. Wafer material is commercially available with different dimensions. The final membrane structure may have a thickness of 5 micrometers to 500 micrometers. The membrane may have a thickness of 10 micrometers to 400 micrometers. The membrane may have a thickness of 30 micrometers to 200 micrometers. The thickness of the membrane structure significantly determines mechanical behaviour of the membrane. Accordingly, the membrane thickness may be chosen depending on the targeted operation frequency and material properties of the membrane. As discussed in more detail below the final thickness of the membrane structure may be adjusted by a selectively etching step during the manufacturing method.

Deposition of the cover layer onto the first surface of the bulk wafer may be performed by any of the above physical vapour deposition (PVD) techniques. The cover layer may be formed from silicon dioxide or silicon nitride.

The cover layer may have a thickness of 0.1 to 5 micrometers. The cover layer may have a thickness of 0.2 to 3 micrometers. The cover layer may have a thickness of 0.3 to 1 micrometer. The thickness of the cover layer and the dimensions of the through-holes defined therein are decisive parameters to control the resulting MMAD (Median Mass Aerodynamic Diameter). Due to considerations with respect to microfluidic properties, small aspect ratios are desired. Thus, cover layers having a small thickness may be preferred. The final choice of the thickness may represent a trade-off with mechanical stability, which may pose a lower limit of the thickness of the cover layer. This lower limit may also depend on the lateral extension of the cover layer and the expected forces applied to the cover layer during usage.

Subsequently through-holes are provided to the cover layer using further MEMS manufacturing techniques. The through-holes through the cover layer may be obtained by a combination of photolithography and etching. For this purpose, the cover layer may be provided with a mask defining the size and position of the through-holes on the cover layer. In a subsequent etching step, the unmasked areas of the cover layer may be etched away, resulting in the cover layer being provided with a plurality of through-holes.

The through-holes formed in the recessed portions may have a diameter of between 0.1 and 4 micrometers. The through-holes formed in the recessed portions may have a diameter of between 0.2 and 3 micrometers.

In a further method step, the second surface of bulk wafer is treated to define recesses therein. These recesses may again be obtained by masking and subsequent etching of the bulk wafer material. Etching of the silicon bulk wafer may be carried out by using sulfur hexafluoride (SF6). Etching of the second surface of the bulk wafer is continued until the bulk waver material is removed from the recesses. The recesses in the second surface are preferably positioned such that they coincide with the positions of the through-holes provided in the cover layer. The combination of the bulk wafer and the cover layer forms a membrane structure having recesses with reduced thickness.

By etching the second surface of the bulk wafer, the final thickness of the membrane structure may be defined. The thickness of the membrane largely determines the vibrational characteristics of the membrane structure.

The two sides of the membrane may also be referred to as the “entrance side” and the “release side” of the membrane. In this regard, the side of the membrane comprising the recesses is the “entrance side”, which in use faces the liquid storage portion, and through which liquid enters into the through-holes. The other side of the membrane forms the “release side” of the membrane, which is the side through which the liquid droplets are released into a downstream mouthpiece chamber. Accordingly, the cover layer comprising the through-holes is provided at the release side of the membrane structure.

In a final step the membrane is cut out from the bulk wafer material. The membrane is cut out to the required dimensions as needed in the application. Typically, the size of the bulk wafers is significantly larger than the required dimension of a membrane, such that in the manufacturing method a plurality of membrane structures may be manufactured in parallel on a single bulk wafer. Such parallel processing allows to manufacture the membranes conveniently in high quantity.

One or more additional layers of material may be applied in manufacturing the membrane. For example, an additional layer may be deposited between the bulk wafer and the cover layer. This additional cover layer may be configured as a selective etching stopper layer. Such selective etching stopper layer may protect the cover layer on the first surface of the bulk wafer, when the recesses are formed at the second surface of the bulk wafer. The selective etching stopper layer may ensure that the etching process for forming the recesses at the second surface of the bulk wafer terminates when the etching fluid has removed the bulk wafer material from the recesses and has reached the selective etching stopper layer. Typically, this is achieved by forming the selective stopper layer from a material that does not dissolve upon contact with the etching agent used for etching the bulk wafer material. The selective etching stopper layer may subsequently be removed by using a different etching solvent.

The thickness of a selective stopper layer may be chosen as suitable. The thickness of a selective stopper layer may range from a few nanometers to 20 micrometers.

In the above example, in which sulfur hexafluoride is used for etching the silicon bulk wafer, the selective etching stopper layer may be formed from any material that does not dissolve upon contact with sulfur hexafluoride. A suitable material in this regard is silicon dioxide. Silicon dioxide does not dissolve upon contact with sulfur hexafluoride, such that the vertical etching process of the bulk wafer will stop, when the etching fluid reaches the silicon dioxide layer. This silicon dioxide layer may later on be removed by another etching agent, such as Hydrogen fluoride (HF) or Trifluoromethane (CHF3).

The technique of using selective etching stopper layer may generally be used to provide the membrane structure with layers having a predefined thickness. In this way also the material defining the thickness of the membrane itself may be deposited as a layer of material being sandwiched between two layers of selective etching stopper material. In this way particularly thin membranes may be provided. In this way also membranes having a well-defined thickness may be provided. The thickness of such membranes may be identical as discussed above with respect to membranes etched from a bulk wafer material. The thickness of such membranes may range from 30 micrometers to 150 micrometers.

Manufacturing the membrane by use of MEMS techniques allows to configure the membrane structure to have desired surface properties that may be beneficial for the aerosolization process. In particular the material properties of the surfaces forming the through-holes and the surfaces that may come into contact with the liquid substrate to be aerosolized may be designed to have such desirous surface properties.

For example, a layer of polycrystalline silicon may be provided below and directly adjacent the cover layer. In this way the entrance side of the through-holes of the cover layer is lined with the layer of polycrystalline silicon. Since polycrystalline silicon is more hydrophilic as compared to single crystalline silicon or silicon nitride, microfluidic flow through the through-holes in enhanced.

The individual method steps discussed above may also be carried out in a different sequence. The skilled person may vary the order of the individual manufacturing steps as deemed suitable.

The present disclosure also relates to a membrane for a vibrating mesh module for use in an aerosol-generating device, wherein the membrane comprises a bulk wafer material that is processed by MEMS manufacturing, and wherein the membrane comprises a plurality of through-holes. The material of the membrane is patterned with an electrically conductive structure. Such electrically conductive track may serve as an intrinsic resistive heater, if a current is running through it.

MEMS manufacturing allows to integrate specific functional features for membranes that are difficult to achieve with conventional membrane manufacturing techniques or that are conventionally only available as add-on features to vibrating mesh modules. According to the present disclosure such functional features may be integrated into the bulk wafer material during the manufacturing process. For example, the bulk wafer material that may serve as the basis for the manufacturing of the membrane may be provided with the pattern of the electrically conductive structure at the beginning of the manufacturing process. Other details of the membrane, such as the application of additional layers or the etching of recessed portions may then be provided at subsequent processing steps.

The electrically conductive structure may be a track made from conductive material. The electrically conductive structure may be a metal track. Such track may be designed such that it extends through parts of the membrane material, which are adjacent to the through-holes, but which are not exposed to the liquid substrate flowing through the membrane. In this way the electrically conductive structure may be protected from coming into direct contact with the liquid material that is to be aerosolized. This may help to prevent degradation of the electrically conductive structure. This may also help to prevent any undesirous effects on the liquid substrate caused by contacting the material of the electrically conductive structure.

In order to be able to run an electric current through the electrically conductive structure, end portions of the electrically conductive structure may reach the outer surface of the membrane structure at a location where there is no liquid flow and where there is no risk for the contact portions to come into contact with the liquid substrate to be aerosolized. Such location may be close to the circumference of the membrane structure. In particular, the membrane may comprise contact pads at its outer circumference to establish an electrical contact to the electrically conductive structure.

In use of an aerosol-generating device, an electric current may be run through the electrically conductive structure, such that the electrically conductive structure acts as an intrinsic heater element. This intrinsic heater element may allow to adjust the operating temperature of the membrane.

The intrinsic heater element may also be used to ensure that the temperature of the liquid substrate flowing through the through-holes has a well-defined temperature. In particular, the heater element may ensure that the temperature of the liquid substrate is rather independent from external conditions such as the ambient temperature. The liquid volume flowing through the through-holes per each cycle is rather small and may amount to only a few microliters per second. In addition, the targeted temperature increase may also be relatively small, and may amount to only a few tens of Kelvin above ambient temperature. Accordingly, the required heating power needed to obtain the temperature increase for the liquid substrate is rather low. The liquid will therefore be heated almost instantaneously upon reaching the membrane and upon flowing though the through-holes. By targeted heating of the circumference of the through-holes of the membrane, this advantageous effect is obtained, while avoiding to have to heat the entire bulk liquid in the reservoir.

The temperature increase of the liquid substrate close to or inside the through-holes, may also result in a local reduction of the viscosity of the liquid substrate. Thus, with a heated membrane also liquid substrates that may have a too high viscosity at room temperature or lower ambient temperatures may be used with aerosol-generating devices comprising such membrane module with intrinsic heater element. This modification generally increases the design space of the liquids useable with such devices. Liquid substrates having increased viscosity may have the beneficial effect that substrate leakage is decreased.

The temperature of the heater element may be kept below the boiling point of the liquid substrate to be aerosolized. The main reason for this is that the primary cause for aerosolization should be the physical interaction of the liquid substrate with the vibrating mesh module. Instead aerosolization caused by heating and evaporation of the liquid substrate and subsequent recondensation should rather be avoided in such systems.

As the typical liquid substrate to be aerosolized by vibrating mesh devices contains a significant amount of water the target temperature of the heater element should not exceed 100 degrees Celsius. The difference between the target temperature of the heater element and ambient temperature should not be higher than 80 degrees Celsius. The difference between the target temperature of the heater element and ambient temperature should not be higher than 60 degrees Celsius. The difference between the target temperature of the heater element and ambient temperature should not be higher than 40 degrees Celsius.

The main advantage of a vibrating mesh module for aerosol-generating devices comprising an intrinsic heater element may be considered to relate to working point stabilization with regard to ambient temperature. By using an intrinsic heater, more uniform aerosolization conditions may be achieved, which may allow for more consistent user experiences.

In addition, a vibrating mesh module for aerosol-generating devices comprising an intrinsic heater element may increase the design range of such devices. Liquid substrates having higher viscosity may be used. The use of liquid substrates may have beneficial effects with respect to handling of the substrate and may in particular prevent substrate to leaking from the supply

The present disclosure also relates to a membrane for a vibrating mesh module for use in an aerosol-generating device, wherein the membrane comprises a bulk wafer material that is processed by MEMS manufacturing, and wherein the membrane comprises a plurality of through-holes. The vibrating mesh module may comprise integrated piezo-elements. Piezo elements may be glued on the membrane during a manufacturing process. Piezo elements that are glued to a membrane are considered as an embodiment of ‘being integrally formed with the membrane’.

MEMS manufacturing may offer the possibility to include piezo-elements during the manufacturing process to a vibrating mesh module. The piezo-elements may be deposited on the membrane module. By incorporating the piezo-elements already during manufacture of the membrane module, attachment of the piezo-elements to the membrane module is facilitated. In conventional manufacturing methods, the piezo-elements have to be clued to the membrane module, which may usually be a cumbersome and fault-prone manufacturing step.

Furthermore, growing the piezo-elements directly to the membrane module allows for a more target specific geometric design of the piezo-elements. The piezo-electric material may be deposited on certain pre-defined locations of the membrane module. Deposition of the piezo-electric material may be obtained by MEMS techniques, such as by sputtering or coating techniques. A mask may be used to achieve the desired lateral geometry of the piezoelectric patches. Alternatively, the desired lateral geometry may also be achieved by providing an overall layer of piezoelectric material and by subsequently removing piezo-electric material from positions where it is not needed. Such removal may again be achieved by masking and etching, or by mechanical removal.

The one or more piezo-elements may be deposited on a contact surface of the membrane structure. The contact surface of the membrane structure may be an annular portion located at a peripheral region of the membrane structure. If deemed to be helpful, the contact portion may also be defined at other surface areas of the membrane structure.

In principle, any material may be used that is suitable to manufacture piezo-elements. Such suitable piezo-electric materials may include lead zirconate titanite, zinc oxide, barium titanite, aluminum nitride, aluminum scandium nitride, lithium niobate, ferroelectric ceramics with perovskite structure and combinations thereof.

A membrane structure as disclosed above, which is additionally provided with one or more integrated piezo-elements may also be referred as a vibrating mesh module. Such vibrating mesh modules may be used in the manufacture of vibrating mesh aerosol-generating devices. Such vibrating mesh modules may be manufactured and sold as interchangeable accessory of such devices. The present disclosure therefore also relates to vibrating mesh modules for aerosol-generating devices and corresponding methods of manufacture thereof.

The present disclosure further relates to a membrane for a vibrating mesh module for use in an aerosol-generating device, wherein electronic circuitry is integrally formed in the material of the vibrating mesh module.

MEMS manufacturing may even offer the possibility to directly include at least parts of the required electronic circuitry in the material of the membrane structure of a vibrating mesh module. In particular the electronic circuitry may be integrally formed in the material of the membrane structure. The electronic circuitry that is included in the membrane structure may include parts of the driver electronics, parts of the microcontroller as well as sensor components.

A vibrating mesh module may comprise one or more piezo-elements. A vibrating mesh module may comprise one or more piezo-elements integrally formed with the membrane structure of the vibrating mesh module. The piezo elements may be included in the membrane structure manufacturing of the membrane with MEMS manufacturing techniques. The piezo-elements may also be glued on the membrane during a manufacturing process. Piezo elements that are glued to a membrane are considered as an embodiment of ‘being integrally formed with the membrane’. In addition thereto, the vibrating mesh module may comprise contact pads for each of the piezo elements. These contact pads may be electrically connected to the piezo-elements and may be formed integrally in the material of the membrane structure. The contact pads may be formed and configured to allow for electric contact with corresponding contact pins of the aerosol-generating device. Such construction may allow for easy exchange of the vibrating mesh module even by the users themselves. To further facilitate such exchange, the aerosol-generating device and the vibrating mesh module may be configured such that the vibrating mesh module may be slid into the aerosol-generating device. Upon sliding the vibrating mesh module into the aerosol-generating device the contact pins of the device may contact the contact pads of the module. By using a plurality of piezo-elements it may be possible to excite different vibration modes of the membrane depending on which of the piezo-element is powered with what frequency. This allows to alter the aerosol forming process and ultimately the characteristics of the resulting aerosol.

The vibrating mesh module may further comprise one or more sensors integrated in the material of the vibrating mesh module or in the material of the membrane structure. Such sensors may include but are not limited to a temperature sensor, a stress-bending sensor, or an acceleration sensor. These sensors could be used for device diagnostics during operation of the aerosol-generating device. These sensors could further be used to provide feedback to the controller of the aerosol-generating device. Since the sensors are provided directly in the material of the vibrating mesh module, they may provide for immediate and direct response from the vibrating mesh module.

The invention further relates to a method of manufacturing a membrane for a vibrating mesh module for use in an aerosol-generating device as described above, wherein the method further comprises the method step of patterning the bulk wafer material with an electrically conductive structure.

The patterning step may be carried out before etching of the bulk wafer material is done. In particular, the patterning step may be carried out as the first method step after providing the bulk wafer material. The pattern of the electrically conductive structure may be such that the conductive structure is provided adjacent to the through-holes of the membrane.

The electrically conductive structure may be a metal track. The electrically conductive structure may be formed such that a contact portion to the electrically conductive portion is provided at the outer periphery of the membrane structure.

The invention further relates to a method of manufacturing a membrane for a vibrating mesh module for use in an aerosol-generating device as described above, wherein the method further comprises the method step of providing one or more piezo-elements, which are integrally formed on a contact surface of the membrane structure.

The piezo elements may be glued on the membrane structure. The piezo elements may be glued on the contact surface of the membrane structure. The piezo elements may be deposited on the contact surface of the membrane structure, by sputtering or by coating techniques. The desired lateral geometry of the piezo elements may be obtained by using a mask.

Alternatively, the desired lateral geometry of the piezo-elements may be obtained by depositing a full layer of piezo-electric material onto the membrane contact surface and subsequently removing the piezoelectric material at positions where the piezo-material is not needed. Forming the piezo-elements already during manufacture of the membrane module may avoid the cumbersome and fault-prone manufacturing step of gluing piezo-elements to the membrane structure, as is done in conventional manufacturing methods for vibrating mesh modules.

The invention further relates to a method of manufacturing a membrane for a vibrating mesh module for use in an aerosol-generating device as described above, wherein the method further comprises the method step of integrally forming in the material of the vibrating mesh module at least parts of the electronic circuitry of the aerosol-generating device.

The electronic circuitry integrally formed in the material of the vibrating mesh module may comprise contact portions for the piezo-elements. The electronic circuitry integrally formed in the material of the vibrating mesh module may comprise one or more sensor elements selected from a temperature sensor, a stress-bending sensor, or an acceleration sensor.

These sensors could be used for device diagnostics during operation of the aerosol-generating device. These sensors could further be used to provide feedback to the controller of the aerosol-generating device. Since the sensors are provided directly in the material of the vibrating mesh module, they may provide for immediate and direct response from the vibrating mesh module.

Below, there is provided a non-exhaustive list of non-limiting examples. Any one or more of the features of these examples may be combined with any one or more features of another example, embodiment, or aspect described herein.

Example 1: A membrane for a vibrating mesh module for use in an aerosol-generating device,
the membrane comprising a plurality of recesses, which recesses define portions of the membrane having reduced thickness,
wherein the portions of the membrane having reduced thickness are provided with one or more through-holes to define perforations of the membrane.

Example 2: The membrane according to example 1, wherein the through-holes have a diameter of between 0.1 and 5 micrometers, preferably of between 0.2 and 3 micrometers.

Example 3: The membrane according to any one of the preceding examples, wherein the through-holes have an aspect ratio of below 3, preferably of below 1.

Example 4: The membrane according to any one of the preceding examples, wherein the through-holes have a circular cross-section.

Example 5: The membrane according to any one of the preceding examples, wherein the membrane has a thickness of 5 micrometers to 500 micrometers, 10 to 400 micrometers, 30 to 200 micrometers.

Example 6: The membrane according to any one of the preceding examples, wherein the recesses defining the portions of the membrane having reduced thickness have a diameter of 5 micrometers to 300 micrometers, preferably of 10 to 200 micrometers, preferably of 15 to 100 micrometers.

Example 7: The membrane according to any one of the preceding examples, wherein a recess comprises from 1 to 1500 through-holes, preferably from 10 to 1000 through-holes.

Example 8: The membrane according to any one of the preceding examples, wherein the membrane comprises density of through-holes of between 10 and 10000 through-holes per squaremillimeter, preferably of between 50 and 5000 through-holes per squaremillimeter.

Example 9: The membrane according to any one of the preceding examples, wherein the recesses have an aspect ratio of below 3.

Example 10: The membrane according to any one of the preceding examples, wherein the membrane comprises a plurality of layers of material.

Example 11: The membrane according to the preceding example, wherein the membrane comprises bulk layer formed from a material provided in the form of a wafer.

Example 12: The membrane according to the preceding example, wherein the wafer material is made of material appropriate for MEMS manufacturing.

Example 13: The membrane according to the preceding example, wherein the wafer material is made of silicon, silicon oxide, silicon nitride, aluminum nitride, or combinations thereof.

Example 14: An aerosol-generating device, comprising a vibrating mesh module for aerosolization of an aerosol-forming substrate, wherein the vibrating mesh module comprises a membrane according to any one of examples 1 to 13.

Example 15: Method of manufacturing a membrane for a vibrating mesh module for use in an aerosol-generating device, the method comprising:
providing a bulk wafer of a first material, the bulk wafer having opposing first and second surfaces,
depositing a cover layer of a second material onto the first surface of the bulk wafer,
providing through-holes to the cover layer using MEMS manufacturing techniques,
etching the second surface of the bulk wafer to define recesses therein,
etching the second surface of the bulk wafer until the bulk wafer material is removed from the recesses,
cutting out the membrane.

Example 16: The method in accordance with example 15, wherein the through-holes are provided to the cover layer using by the steps of
masking the cover layer to define locations and dimensions of through-holes of the membrane to be manufactured,
etching the cover layer to create the through-holes in the cover layer.

Example 17: The method in accordance with example 15 or 16 further comprising:
depositing an additional layer between the bulk wafer and the cover layer, wherein the additional layer is configured as a selective etching stopper layer.

Example 18: The method in accordance with example 17 further comprising:
depositing a further selective etching stopper layer between the bulk wafer and the cover layer, and
depositing a further layer of material between the two selective etching stopper layers, wherein the thickness of this further layer of material determines the thickness of membrane to be manufactured.

Example 19: The method in accordance with any one of examples 15 to 18, wherein the material of the bulk wafer and/or of the further layer of material is selected of silicon, silicon oxide, silicon nitride, aluminum nitride, or combinations thereof.

Example 20: The method in accordance with any one of examples 15 to 18, further comprising:
depositing an additional layer of material below the cover layer, wherein the additional layer allows to define adjust surface properties of the material defining the through-holes of the membrane.

Example 21: The method in accordance with example 20, wherein the additional layer is a layer which is more hydrophilic than the material of the cover layer.

Example 22: The method in accordance with any one of examples 20 or 21, wherein the additional layer is a layer of polycrystalline silicon.

Example 23: A membrane for a vibrating mesh module for use in an aerosol-generating device,
the membrane being made from material appropriate for MEMS manufacturing,
the membrane comprising a plurality of through-holes, wherein the material of the membrane is patterned with an electrically conductive structure.

Example 24: A membrane in accordance with example 23, wherein the electrically conductive structure is configured to extend through parts of the membrane material.

Example 25: A membrane in accordance with any one of examples 23 or 24, wherein the electrically conductive structure is provided adjacent to the through-holes, but not exposed to liquid flow path defined through the through-holes in the membrane.

Example 26: A membrane in accordance with any one of examples 23 or 25, wherein the membrane comprises contact pads at its outer circumference to establish an electrical contact to the electrically conductive structure.

Example 27: A membrane in accordance with any one of examples 23 or 26, wherein in use an electric current is run through the electrically conductive structure, such that the electrically conductive structure acts as a heater element that allows to adjust the operating temperature of the membrane.

Example 28: A membrane in accordance with any one of examples 1 to 14 or 23 to 27, further comprising one or more piezo-elements deposited on a contact surface of the membrane structure.

Example 29: A membrane in accordance with the preceding example, wherein the contact surface is an annular portion located at a peripheral region of the membrane structure.

Example 30: A membrane in accordance with any one of examples 28 to 29, wherein the material of the piezo-elements is selected from lead zirconate titanite, zinc oxide, barium titanite, aluminum nitride, aluminum scandium nitride, lithium niobate, or ferroelectric ceramics with perovskite structure.

Example 31: A vibrating mesh module for use in an aerosol-generating device, the vibrating mesh module comprising:
a membrane in accordance with any one of examples 1 to 14 or 23 to 27, further comprising one or more piezo-elements deposited on a contact surface of the membrane structure.

Example 32: A membrane in accordance with any one of examples 1 to 14 or 23 to 30, wherein electronic circuitry is integrally formed in the material of the vibrating mesh module.

Example 33: A membrane in accordance with the preceding examples, wherein the electronic circuitry comprises electronic contact structures for the one or more piezo-elements of the membrane structure.

Example 34: A membrane in accordance with any one of examples 32 to 33, wherein the electronic circuitry comprises one or more sensor elements.

Example 35: A membrane in accordance with the preceding example wherein the one or more sensor elements are selected from a temperature sensor, a stress-bending sensor, or an acceleration sensor.

Example 36: A membrane in accordance with any one of examples 34 to 35, wherein the sensors are used for device diagnostics during operation or to provide feedback to a controller of the aerosol-generating device.

Example 37: The method according to any one of examples 15 to 22, the method further comprising:
patterning the bulk wafer material with an electrically conductive structure.

Example 38: The method according to example 37, wherein the electrically conductive structure is a metal track.

Example 39: The method according to any one of examples 37 or 38, wherein the patterning step is carried out before etching the bulk wafer material.

Example 40: The method according to any one of examples 15 to 22, wherein one or more piezo elements are integrally formed on a contact surface of the membrane structure.

Example 41: The method according to the preceding example, wherein the piezo elements are deposited on the contact surface of the membrane structure, by sputtering or by coating techniques.

Example 42: The method according to the preceding example, wherein the desired lateral geometry of the piezo elements is obtained by using a mask.

Example 43: The method according to the preceding example, wherein the desired lateral geometry of the piezo elements is obtained by depositing a full layer of piezoelectric material onto the membrane contact surface and subsequently removing the piezoelectric material at positions where it is not needed.

Example 44: The method according to any one of examples 15 to 22, wherein electronic circuitry is integrally formed in the material of the vibrating mesh module during manufacture.

Example 45: The method in accordance with the preceding example wherein the electronic circuitry comprises one or more sensor elements selected from a temperature sensor, a stress-bending sensor, or an acceleration sensor.

Example 46: The method in accordance with the preceding example wherein the sensors are used for device diagnostics during operation or to provide feedback to a controller of the aerosol-generating device.

Features described in relation to one embodiment may equally be applied to other embodiments of the invention.

The invention will be further described, by way of example only, with reference to the accompanying drawings in which:

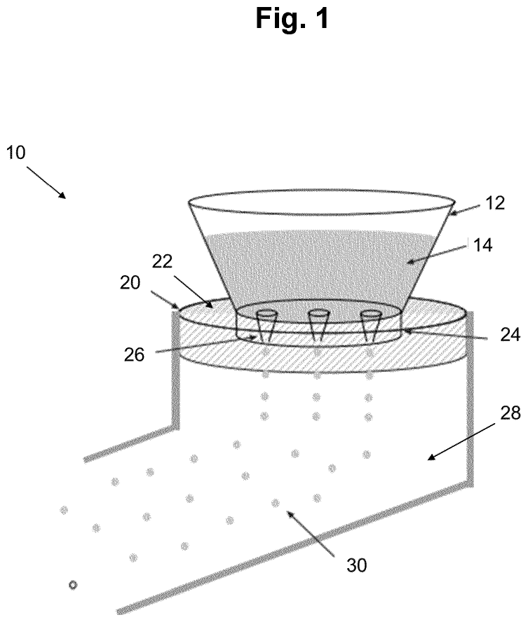

FIG. 1 shows a schematic design of a conventional vibrating mesh nebulizer;

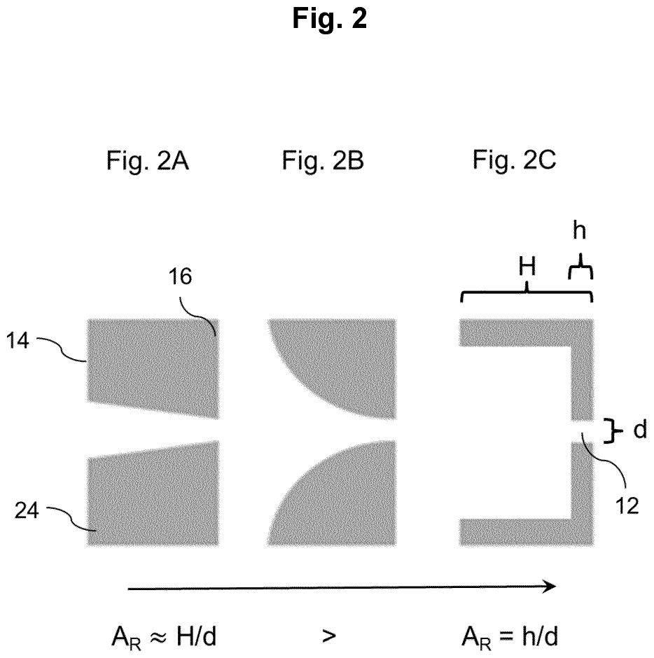

FIG. 2 shows geometries of through-holes in membranes obtainable by different manufacturing methods;

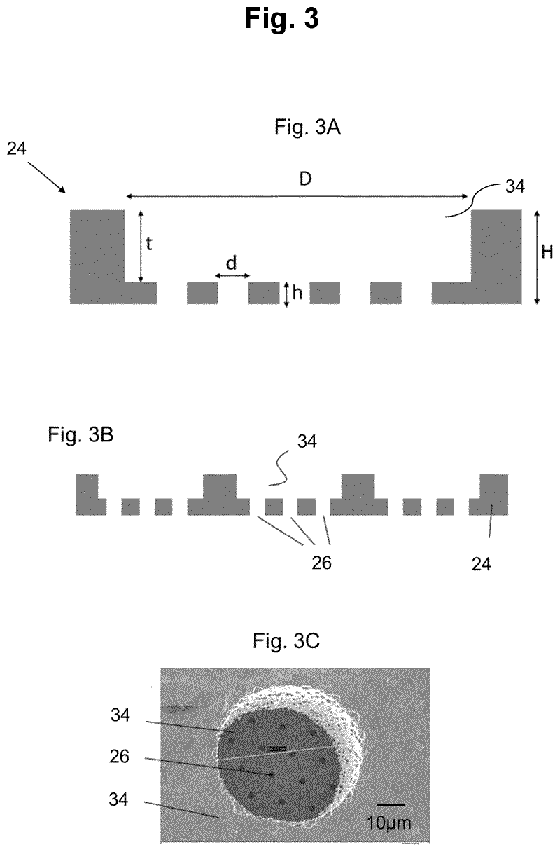

FIG. 3 shows detailed views of a MEMS membrane;

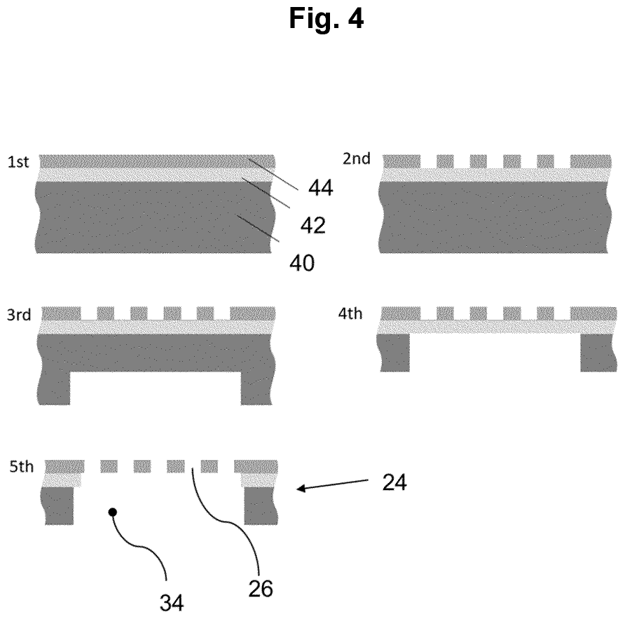

FIG. 4 illustrates the method steps to manufacture a MEMS membrane;

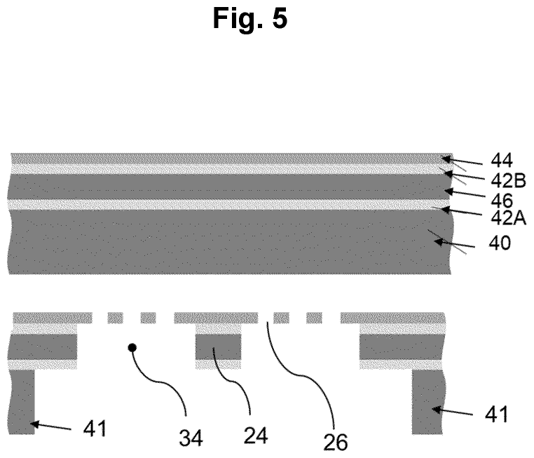

FIG. 5 shows a modification of the method of FIG. 4;

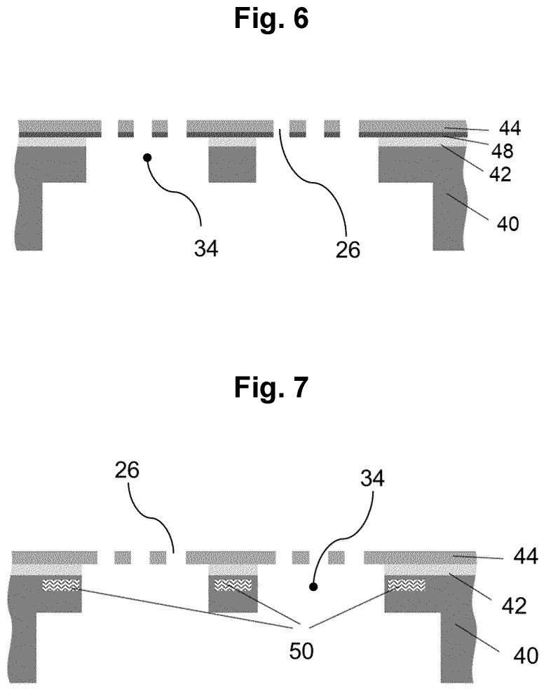

FIG. 6 shows a MEMS membrane provided with a hydrophilic layer;

FIG. 7 shows a MEMS membrane provided with an intrinsic heater;

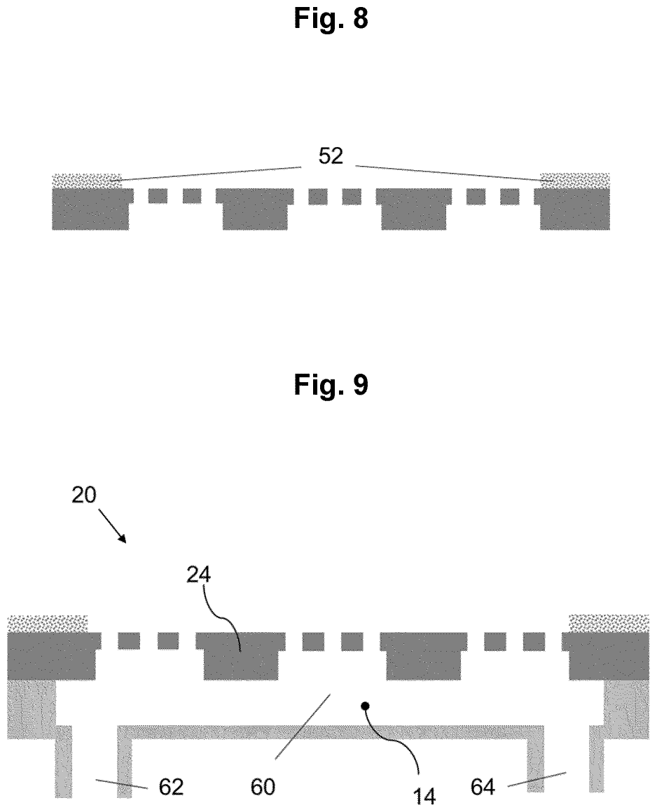

FIG. 8 shows a MEMS membrane provided with integrated piezo-elements; and

FIG. 9 shows a VM module connected to an upstream liquid flow chamber.

FIG. 1 shows an aerosol-generating device 10 that may also be referred to as a nebulizer. The nebulizer comprises a liquid storage portion 12 holding a supply of liquid substrate 14 to be aerosolized. In direct contact with the liquid substrate 14 there is provided a vibrating mesh module 20, which comprises an annular piezo-element 22 encompassing a circular mesh. The mesh is configured as a membrane 24 comprising through-holes 26. The through-holes 26 in the membrane 24 have a conical structure, with the largest cross-section of the cone in contact with the liquid drug. By vibration of the membrane 26 the liquid substrate 14 is pumped through the through-holes 26 and sprayed into a mouthpiece chamber 28. During inhalation, ambient air crosses the mouthpiece chamber 28 to transport the generated aerosol 30 to the user.

The size of the through-holes 26 at the exit side of the membrane 24 as well as the aspect ratio of the through-holes 26 are key parameters to determining the aerosolization process. FIG. 2 shows enlarged schematic views of membranes 24 comprising through-holes 26. The membranes 24 are depicted in descending order from left to right according to the aspect ratio AR of the through-holes 26 thereof. This order is indicated by the arrow pointing from the left hand side to the right hand side in FIG. 2. Each membrane 24 has an identical thickness H and the through-holes 26 have an identical minimum diameter d at the exit side 32 of the membrane 24. The through-holes 26 in the membranes 24 are obtained by different manufacturing methods, wherein each manufacturing method leads to through-holes 26 having different aspect ratios. In general, an aspect ratio AR of a through-hole 26 is defined by the its length divided by its diameter.

The membrane 24 depicted in FIG. 2A is a membrane 24 made of stainless steel. The through-holes 26 therein are created by laser drilling. Laser drilling leads to through-holes 26 having a conical shape with small taper angle. The through-holes 26 obtained in this way have a comparably large aspect ratio AR. The aspect ratio AR of these through-holes may be approximated by the quotient H/d, wherein H is the thickness of the membrane 24 and d is the minimum diameter of the through-hole 26 at the exit end of the membrane 24.

The membrane 24 depicted in FIG. 2B is a membrane 24 made of a nickel-cobalt alloy. The through-holes 26 therein are created by lithography and subsequent electroplating. In a first step lithography is used to create in the membrane 26 a pattern of a plurality of through-holes 26 with a comparatively large diameter. Subsequently, the entire membrane 24 undergoes electroplating to deposit material on the membrane surface thereby shrinking the diameter of the through-holes 26. This results in through-holes 26 having a funnel-like shape. These through-holes 26 have a somewhat smaller aspect ratio AR and would thus be preferred over laser drilled through-holes 26.

The membrane 24 depicted in FIG. 2C is a membrane 24 according to the present disclosure. The membrane 24 comprises a recess 34 in which the through-hole 26 is provided. The remaining thickness h of the membrane 24 in the recess 34 is considerably smaller than the overall thickness H of the membrane 24. Accordingly, the aspect ratio AR of the through-holes 26 of such membrane 24 may be expresses as the quotient h/d, which is much smaller than the aspect ratios AR of the through-holes 26 of the membranes 24 depicted in FIGS. 2A and 2B.

In FIG. 3 a portion of a membrane 24 according to the present disclosure is depicted in more detail. FIG. 3A shows an enlarged view of a recess 34 of such membrane 24. The membrane 24 is made from silicon and has an overall thickness H. The recess 34 has a circular cross section with a diameter D and a depth t. The thickness h of the recessed portion of the membrane 24 is determined as h=H−t. A plurality of through-holes 26 is provided at the recesses 34. These through-holes 26 also have a circular cross section and have a diameter d which is much smaller than the diameter D of the recesses 34.

As indicated in FIG. 3B a membrane 24 may comprise a plurality of recesses 34 while each of the recesses 34 in turn may comprise a plurality of through-holes 26 through which the liquid substrate 14 is released into a mouthpiece chamber 28 of the aerosol-generating device 10.

FIG. 3C shows an electron-microscopical image of a portion of the membrane comprising a recess 34. The membrane has a thickness of about 100 micrometers. The circular recess 34 has a diameter D of about 50 micrometers. The remaining thickness h of the recess 34 amounts to 1 micrometer. The recess comprises 14 circular through-holes 26 which are evenly distributed in the recess 34 in a hexagonal pattern. The diameter d of the through-holes 26 amounts to about 2 micrometers. Thus, the aspect ratio of the through-holes of the membrane shown in FIG. 3C amounts to about 0.5.

A membrane 24 as depicted in FIG. 3 may be manufactured by a sequence of manufacturing steps involving various MEMS processing techniques. A suitable manufacturing method is illustrated in FIG. 4. In a first step a bulk wafer 40 made of silicon is provided as a base substrate. On top of a first surface of the bulk wafer 40 two layers 42, 44 of material are formed by thin film deposition. Layer 42 is formed from silicon dioxide and is used as a selective etching stopper layer. On top of layer 42, there is deposited a layer 44, which is formed from silicon nitride. Layer 44 serves as cover layer of the membrane 24.

In a second step, a mask defining the locations and the diameters of the through-holes 26 is applied to the cover layer 44 by lithography. The cover layer 44 is then dry etched with carbon tetrafluoride (CF4) to locally remove material of the cover layer 44 and to create through-holes 26 in the silicon nitride cover layer 44.

In a third step, recesses are defined in the second surface or the back side of the silicon bulk wafer 40. For this purpose, a mask defining the position and the cross section of the recesses is applied by lithography to the second surface of the bulk wafer 40. Subsequently, the recesses 34 are formed by etching the second surface of the silicon bulk wafer 40 with sulfur hexafluoride (SF6).

In a fourth step the complete second surface of the silicon bulk wafer 40 is etched further until the bulk waver material is removed from the recesses 34. Due to the use of the selective etching stopper layer 42 made from silicon dioxide, which does not dissolve upon contact with sulfur hexafluoride, etching of the recesses 34 stops once the silicon dioxide layer 42 is reached. In this step, the bulk wafer material surrounding the recesses 34 is subjected to etching until the bulk wafer has reached a desired thickness. The thickness of the remaining silicon bulk wafer material surrounding the recesses 34 defines the final thickness of the membrane 24.

In a fifth step, the silicon dioxide layer 42 is removed by etching with hydrogen fluoride (HF). By this etching step the accessible portions of the silicon dioxide layer 42 in the recesses 34 are removed, whereby the through-holes 26 in the cover layer 44 are connected to the recesses 34 in the bulk wafer 40. In a last step (not shown) the membrane 24 may be cut out in the desired size from the bulk wafer 40.

With the method illustrated in FIG. 4 a membrane 24 comprising through-holes 26 is obtained by making use of a sequence of MEMS techniques. With these techniques a membrane 24 is obtained that comprises well-defined through-holes 26 having diameters that are significantly smaller than those obtainable by currently used manufacturing techniques. The membrane 24 allows for sufficient liquid throughput and has a flow resistance that makes the membrane 24 suitable for use in a vibrating mesh module 20 for aerosol-generating devices 10.

In FIG. 5 a further membrane 24 is depicted in which an additional layer of silicon 46 is arranged between two selective etching stopper layers 42A, 42B. The membrane 24 is generally obtained by a similar method as described with FIG. 4 with the exception that in the first manufacturing step a sequence of selective etching stopper layer 42A, silicon layer 46, selective etching stopper layer 42B and cover layer 44 is deposited on the silicon bulk wafer 40 as depicted in the top view of FIG. 5. The final structure of the membrane 24 is depicted in the lower view of FIG. 5. The thickness of the actual membrane 24 is now defined by the additional silicon layer 46 and the recesses 34 are formed in this silicon layer 46. The bulk wafer 40 is largely etched away and only side walls 41 remain as lateral frame structures supporting the membrane 24 extending therebetween. These frame structures and the recesses 34 are again obtained by sequences of masking and etching as described above. In FIG. 5 only two recesses 34 are shown, whereas a membrane 24 may of course comprise a much higher number of recesses 34. In the same way as described with FIG. 4, each recess 34 is again covered by cover layer 44. The cover layer 44 comprises again through-holes 26 through which the liquid substrate 14 is sprayed into the mouthpiece chamber 28 of the aerosol-generating device 10. By using the additional silicon layer 46 between the two selective etching stopper layers 42 A,B a membrane 24 with a well-defined thickness is obtained. Thus, this method allows for a more precise control of the membrane thickness.

By an appropriate choice of materials, the membrane structure may be configured to have desired surface properties that may be beneficial for the aerosolization process. In this regard, FIG. 6 depicts a membrane structure in which an additional layer 48 of polycrystalline silicon is provided below and directly adjacent to the cover layer 44. In this way the surface of the cover layer 44 which is in contact with the liquid substrate 14 stored in the liquid supply portion 12 is lined with the layer 48 of polycrystalline silicon. Since polycrystalline silicon is more hydrophilic as compared to single crystalline silicon or silicon nitride, microfluidic flow through the through-holes 26 in enhanced.

FIG. 7 shows a further modification of the membrane structure depicted in FIG. 4. The bulk wafer 40 forming the base substrate of the membrane 24 is patterned with an electrically conductive structure 50. Patterning of the membrane material may be carried out by using any suitable doping method known to the skilled person. In the method of FIG. 5 the electrically conductive structures 50 may also be formed during vapor deposition of the material layer forming the membrane 24. As depicted in FIG. 7, the electrically conductive structures 50 extend through parts of the membrane material adjacent to the recesses 34 and through-holes 26, but are not exposed to the liquid substrate 14 flowing through the membrane 24. Such electrically conductive structures 50 may form intrinsic resistive heater elements. By running an electric current through these electrically conductive structures 50, the temperature of the membrane 24 and of the liquid substrate 14 to be aerosolized may be adjusted.

MEMS techniques also allow to deposit further functional layers or components on the membrane structure. In this regard, FIG. 8 shows a yet further modification of the previously described membrane structures. The membrane structure is provided with integrated piezo-elements 52 that are formed on an annular contact area 54 of the membrane structure.

The piezo-electric material, in this case lead zirconate titanite, is deposited on top of the membrane structure during the manufacturing process of the membrane 24. As depicted in FIG. 8 the piezo-electric material is deposited in an annually extending contact area 54, which is located at peripheral region of the membrane structure 24. Deposition of the piezo-electric material is carried out by coating techniques, including a masking step so as to achieve the desired lateral geometry of the piezo-elements 52. Forming the piezo-elements 52 integrally with the membrane structure 24 makes manufacturing of vibrating mesh modules 20 easier, compared to conventional manufacturing methods. In conventional manufacturing methods, separately provided piezo-elements 52 have to be glued to a membrane module, which usually is a cumbersome and fault-prone manufacturing step.

The membrane structure 25 may further be provided with contact portions for electrically contacting the piezo-elements 52 to a controller of the aerosol-generating device 10. Such membrane structure as depicted in FIG. 8, which is additionally provided with one or more integrated piezo-elements 52 and electrical contact portions may also be referred to as a vibrating mesh module 20. Such vibrating mesh modules 20 may be used in manufacture of vibrating mesh aerosol-generating devices 10. The vibrating mesh modules 20 may be manufactured as interchangeable accessory of such aerosol-generating devices 10 and may be configured to be exchanged and replaced by the users themselves.

FIG. 9 shows a vibrating mesh module 20 of FIG. 8 connected to an upstream liquid flow chamber 60. The liquid flow chamber 60 is configured to provide liquid substrate 14 to the entrance side of the membrane structure 24. The liquid flow chamber 60 is in fluid communication with a liquid supply portion (not shown) of the aerosol-generating device. The fluid communication is established via an inlet 62 and an outlet 64 which allows the liquid substrate 14 to freely circulate between the flow chamber 60 and the liquid supply portion. This configuration ensures that the entrance side of the membrane 24 is always in contact with the liquid substrate 14. This configuration further ensures that excess liquid substrate 14 that is not dispersed through the through-holes, is fed back into the liquid supply portion. With this configuration the liquid substrate 14 is not pressed against or through the through-holes of the membrane 24. This configuration allows for enhanced reproducibility of the aerosol formation and avoids unwanted leakage of liquid substrate 14.

1.-15. (canceled)

16. A membrane for a vibrating mesh module for an aerosol-generating device, the membrane comprising:
a plurality of recesses defining portions of the membrane having reduced thickness, wherein the portions of the membrane having reduced thickness are provided with one or more through-holes defining perforations of the membrane; and
one or more piezo elements integrally formed therewith,
wherein the through-holes have an aspect ratio of below 1, and
wherein the through-holes have a diameter of between 0.2 micrometer and 3 micrometers.

17. The membrane according to claim 16, further comprising a density of through-holes of between 10 and 10000 through-holes per square millimeter.

18. The membrane according to claim 16, further comprising a density of through-holes of between 50 and 5000 through-holes per square millimeter.

19. The membrane according to claim 16, wherein the recesses have an aspect ratio of below 3.

20. The membrane according to claim 16, further comprising a plurality of layers of material.

21. The membrane according to claim 20, further comprising a bulk layer formed from a material provided in the form of a wafer.

22. The membrane according to claim 21, wherein the wafer material is made of silicon, silicon oxide, silicon nitride, aluminum nitride, or combinations thereof.

23. An aerosol-generating device, comprising a vibrating mesh module for aerosolization of an aerosol-forming substrate, wherein the vibrating mesh module comprises a membrane according to claim 16.

24. A method of manufacturing a membrane for a vibrating mesh module for an aerosol-generating device, the method comprising:
providing a bulk wafer of a first material, the bulk wafer having opposing first and second surfaces;
depositing a cover layer of a second material onto a first surface of the bulk wafer;
providing through-holes to the cover layer using MEMS manufacturing techniques;
etching the second surface of the bulk wafer to define recesses therein;
etching the second surface of the bulk wafer until the bulk wafer material is removed from the recesses; and
cutting out the membrane,
wherein the membrane comprises one or more piezo elements integrally formed therewith,
wherein the through-holes have an aspect ratio of below 1, and
wherein the through-holes have a diameter of between 0.2 micrometer and 3 micrometers.

25. The method according to claim 24, wherein the through-holes are provided to the cover layer using by the steps of
masking the cover layer to define locations and dimensions of through-holes of the membrane to be manufactured, and
etching the cover layer to create the through-holes in the cover layer.

26. The method according to claim 24, further comprising patterning the bulk wafer material with an electrically conductive structure.

27. A membrane for a vibrating mesh module for an aerosol-generating device,
the membrane being made from material appropriate for MEMS manufacturing,
the membrane comprising a plurality of recesses defining portions of the membrane having reduced thickness,
wherein the portions of the membrane having reduced thickness are provided with one or more through-holes to define perforations of the membrane,
wherein the material of the membrane is patterned with an electrically conductive structure,
wherein the through-holes have an aspect ratio of below 1, and
wherein the through-holes have a diameter of between 0.2 micrometer and 3 micrometers.

28. The membrane according to claim 27, wherein the electrically conductive structure is configured to extend through parts of the membrane material.

29. The membrane according to claim 27, further comprising one or more piezo elements deposited on a contact surface of the membrane structure.

30. A vibrating mesh module for an aerosol-generating device, the vibrating mesh module comprising:
a membrane according to claim 27; and
one or more piezo elements deposited on a contact surface of the membrane structure.
